(19) United States
(12) Patent Application Publication
(10) Pub. No.: US 2026/0088186 A1
(43) Pub. Date: Mar. 26, 2026

(54) Generating Positioning Information for a Patient Examination Protocol

(71) Applicant: Siemens Healthineers AG, Forchheim (DE)

(72) Inventors: Dominik Paul, Bubenreuth (DE); Fabian Wagner, Erlangen (DE)

(73) Assignee: Siemens Healthineers AG, Forchheim (DE)

(21) Appl. No.: 19/333,480

(22) Filed: Sep. 19, 2025

(30) Foreign Application Priority Data
Sep. 20, 2024 (DE) 10 2024 209 049.2

(51) Int. Cl. G16H 70/20; A61B 5/00; G16H 30/40

(52) U.S. Cl. CPC G16H 70/20; A61B 5/70; G16H 30/40

(57) ABSTRACT

A computer-assisted method for generating positioning information for an examination protocol for the examination of a patient, including: providing image data of an overview recording of a patient; providing an evaluation model configured, based on the image data, to generate positioning information for an examination protocol for the examination of the patient; and generating positioning information for the examination protocol by applying the evaluation model to the image data, wherein the positioning information is usable in the examination protocol for the examination of the patient.

### TECHNICAL FIELD

The present disclosure relates to a computer-assisted method for provision of positioning information for an examination protocol for the examination of a patient, to a system for provision of positioning information for an examination protocol for the examination of a patient, and to a corresponding computer program element.

### BACKGROUND

In medical engineering, different slice imaging methods are known, such as, for example, magnetic resonance tomography and computed tomography. A magnetic resonance tomography device is an imaging apparatus that, for mapping an examination object, aligns nuclear spins of the examination object with a strong external magnetic field and excites them by a magnetic alternating field for precession about said object. The precession of the spins from this excited state into a state with lower energy in its turn generates an alternating magnetic field as a response, which is received by antennas. With the aid of magnetic gradient fields, a spatial encoding is impressed on the signals, which then makes possible an assignment of the received signal to a volume element. The received signal is then evaluated, and a three-dimensional imaging representation of the examination object can be provided.

A computed tomography device is an imaging apparatus that, for mapping of an examination object, “samples” said object by a mostly rotating x-ray tube from all directions while the examination object is being pushed forward through an opening, also known as a gantry, of the computed tomography device. Tissue with strong x-ray absorption appears bright and tissue with lower x-ray absorption appears dark. The received signal is then evaluated, and a three-dimensional imaging representation of the examination object can be provided.

During these examinations, the operating personnel are to be assisted as far as possible by automated examination sequences. In particular, automated examination protocols should be carried out. In an examination protocol, the parameters are defined, for example, for which a specific region of a patient is to be examined. In such cases, the positioning of the examination protocol is to occur automatically where possible. A positioning of the examination protocol is to be understood in this case as where on the patient or for which region of the patient the examination is to be undertaken by the parameters defined for the examination protocol.

Such an automated positioning of the examination protocol is especially problematic in such cases where the anatomy to be examined is present twice on the patient, as is the case with knees, hip regions, and the like.

In this connection, it has now emerged that there is a need to simplify the positioning of an examination protocol or to be able to carry out the positioning of an examination protocol in an automated manner, where possible. In particular, a need exists to simplify the positioning of an examination protocol when the patient has two anatomical areas to be examined.

### SUMMARY

An object of the present disclosure is therefore to provide a solution in order to simplify a positioning of an examination protocol or to be able to carry out the positioning of an examination protocol in an automated manner where possible. In particular, an object of the present disclosure is to simplify the positioning of an examination protocol when the patient has two of the anatomy to be examined.

These, and other objects that the reader will also encounter when reading the following description or that can be recognized by the person skilled in the art, are achieved by the subject matter of the independent claims. The dependent claims develop the central idea of the present disclosure in an especially advantageous way.

A first aspect of the present disclosure relates to a computer-assisted method for provision of positioning information for an examination protocol for the examination of a patient, comprising: provision of image data of an overview recording of a patient; provision of an evaluation model, which is configured, based on the image data provided, to provide positioning information for an examination protocol for an examination of the patient; and provision of positioning information for an examination protocol for the examination of the patient by application of the evaluation model and of the image data provided.

In other words, the present disclosure proposes to evaluate an overview recording, known as a scout recording, in order to be able to provide positioning information for an examination protocol. In an example, the evaluation model is configured in this case, when there are two of an anatomy of a patient present, for example, the left knee and the right knee, to establish that knee on which the examination protocol is to be positioned. In such an example, positioning information can comprise lateral information, with which an assignment to one of the two anatomies is possible. In the sense of the present disclosure, positioning information is generally to be understood in this case as any item of information that allows an examination protocol to be positioned.

The term “provision” is to be understood in broad terms here and stands for any provision, receipt, interrogation, measurement, computation, definition, transmission of data, but is not restricted to these, however. Data can be provided by a user via a user interface, displayed/shown to a user via a display, and/or received from other devices, requested by other devices, measured by other devices, computed by other devices, determined by other devices, and/or transmitted by other devices.

The term “data” is to be understood in broad terms here and stands for any kind of digital data. Data can comprise individual numbers/numerical values, a plurality of numbers/numerical values, a plurality of numbers/numerical values that are arranged in a list, two-dimensional maps, or three-dimensional maps. The term “data” is not restricted to this, however. The data can also be provided in different formats.

In one exemplary aspect, the evaluation model is furthermore configured to evaluate the image data for an asymmetrical arrangement of the patient in relation to a reference axis or a reference plane in order to provide the positioning information for the examination protocol. The evaluation of the arrangement of a patient by comparison with a reference axis, for example the Z axis of a magnetic resonance tomography device or a computed tomography device, is based on the knowledge that the region of a patient to be examined will in practice be arranged closer to the center, since the image quality/data quality to be expected there is at its highest. Thus, with a doubled anatomy of a patient, that anatomy is to be established that is arranged closer to the Z axis on the patient couch. In the example of a magnetic resonance tomography device, the reference system is typically aligned to the B0 field magnet. In such a reference system, the Z axis corresponds to an axis of symmetry of the B0 field magnet in the preferred direction of the B0 field, wherein the axes are preferably provided orthogonal to one another, and the X axis is preferably arranged horizontally, and the Y axis is preferably arranged vertically.

In one exemplary aspect, the evaluation model is furthermore configured in order to divide the image data of the overview recording with reference to the reference axis or reference plane into image data of a first region and image data of a second region. In the example of a doubled anatomy of a patient, the first region captures a first anatomy, and the second region captures the second anatomy. If, for example, a doubled anatomy involves a left and a right knee, the first region should, for example, comprise the left knee and the second region the right knee. It is preferred in this case for one anatomy to be exclusively comprised by one region.

In one exemplary aspect, the evaluation model is furthermore configured in order to provide first projection data of the first region on an X axis and second projection data of the image data of the second region on the X axis. Such projection of the image data on the X axis enables an intensity distribution along the X axis to be provided. The slice image of the overview recording. This enables so-called empty pixels, i.e., pixels that do not show any tissue, to be identified and removed more easily.

In one exemplary aspect, the evaluation model is furthermore configured in order to provide first histogram data of the first projection data and second histogram data of the second projection data, wherein the histogram data is preferably gray value histogram data. The histogram data provides the distribution of the respective brightness values of the two regions or projection data. Where the histogram data is to be visualized, said data can be visualized in the form of bar diagrams, for example. The gray values are listed on the horizontal axis in this case, and the frequencies of the respective brightness values are parallel to the vertical axis.

In one exemplary aspect, the evaluation model is furthermore configured in order to provide first focus data of the first histogram data and second focus data of the second histogram data. In one exemplary aspect, the evaluation model is furthermore configured to provide the positioning information for the examination protocol at least by a comparison of the first focus and the second focus. In an example, the first focus of the first histogram data and the second focus of the second histogram data are provided by the following formula:

$$S = 1 / n {\sum }_{k = 1}^{n} {W}_{k} * {B}_{k} ,$$

wherein S represents the focus of the histogram data, n represents total number of entries in the histogram data, Bₖ represents number of entries in bin k, and Wₖ represents value of the bin k.

In one exemplary aspect, the evaluation model comprises at least one trained algorithm, which is configured, at least based on the image data, to provide positioning information for the examination protocol. The term “trained algorithm” used here is to be understood in broad terms in this case. The algorithm can be a machine learning algorithm. The algorithm can comprise decision trees, naive Bayesian classifications, nearest neighbors, neural networks, convolutional networks, or recurrent neural networks, transformers, generative adversarial networks, support vector machines, linear regression, logistical regression, random forest, gradient boosting algorithms, and/or a diffusion model. Such an algorithm, in particular a machine learning algorithm, is referred to as intelligent because it can be trained. The algorithm can be trained with aid of training datasets. A training dataset comprises training input data and corresponding training output data. Within the framework of the present disclosure, the training data can, for example, comprise the image data of an overview recording of a patient and at least one assignment of a corresponding item of positioning information for an examination protocol. The image data of an overview recording of a patient can involve the training input data. The positioning information for an examination protocol can involve the training output data. The training output data of a training dataset is the result that was expected from the algorithm when it receives the training input data of the same training dataset as input. The deviation between this expected result and the result actually achieved by the algorithm is considered and for example, assessed with the aid of a loss function. This loss function is used as feedback for the adjustment of the parameters of the internal processing chain of the algorithm. For example, the parameters can be adapted with the optimization objective of minimizing the values of the loss function that are produced when all training input data is fed into the algorithm, and the result is compared with the corresponding training output data. The result of this training is that the algorithm with a relatively small number of training datasets, as “ground truth”, is capable of fulfilling its task well for a number of input datasets higher by many orders of magnitude.

In one exemplary aspect, the positioning information comprises an item of lateral information for a doubled anatomy present on a patient. In one example, the lateral information involves information as to which of the two anatomies of a patient the examination protocol is to be positioned on. For example, whether the examination protocol should be positioned on a left knee on a right knee.

In one exemplary aspect, the method furthermore comprises:
provision of control data for controlling a magnetic resonance tomography device or a computed tomography device based on the positioning information provided. In an example, the positioning information provided can be employed directly for positioning of the examination protocol. In a further example, the positioning information provided can be suggested to an operator for confirmation.

A further aspect of the present disclosure relates to as system for provision of positioning information for an examination protocol for the examination of a patient, comprising: a processing circuit; a storage medium; and a data line; wherein the storage medium comprises a computer program with instructions that, when the program is executed, cause the processing circuit to carry out the present method described, wherein the data interface is configured to receive the image data of the overview recording of the patient for the evaluation model.

A further aspect of the present disclosure relates to a computer program element comprising instructions that, during execution on computer devices of a computer environment, are configured to carry out the steps of the method described here in a system for provision of positioning information for an examination protocol for the examination of a patient.

A further aspect of the present disclosure relates to a use of image data of an overview recording of a patient; and/or of an evaluation model, which is configured, based on the image data provided, to provide positioning information for an examination protocol for the examination of the patient; in a method described here for provision of positioning information for an examination protocol for the examination of a patient. A use of the image data and/or of the evaluation method is to be understood as the use of the image data and/or of the evaluation method in the method disclosed here.

### BRIEF DESCRIPTION OF THE DRAWINGS

All forms of aspects described herein can be combined with one another, unless explicitly specified otherwise. Further features, advantages, and possible applications of the present disclosure emerge from the description, the exemplary aspect, and the figures given below. In the figures:

FIG. 1 shows a flow diagram of an exemplary aspect of a computer-assisted method for provision of positioning information for an examination protocol for the examination of a patient;

FIG. 2 shows an exemplary aspect of a disclosed system;

FIG. 3 shows an axial section through image data with two knees;

FIG. 4 shows image data for a first region, a sub-volume on the right with a right knee;

FIG. 5 shows image data for a second region, a sub-volume on the left with a left knee;

FIG. 6 shows projection data of the image data shown in FIGS. 4 and 5;

FIG. 7 shows histogram data for the sub-volume to the right; and

FIG. 8 shows histogram data for the sub-volume to the left.

### DETAILED DESCRIPTION

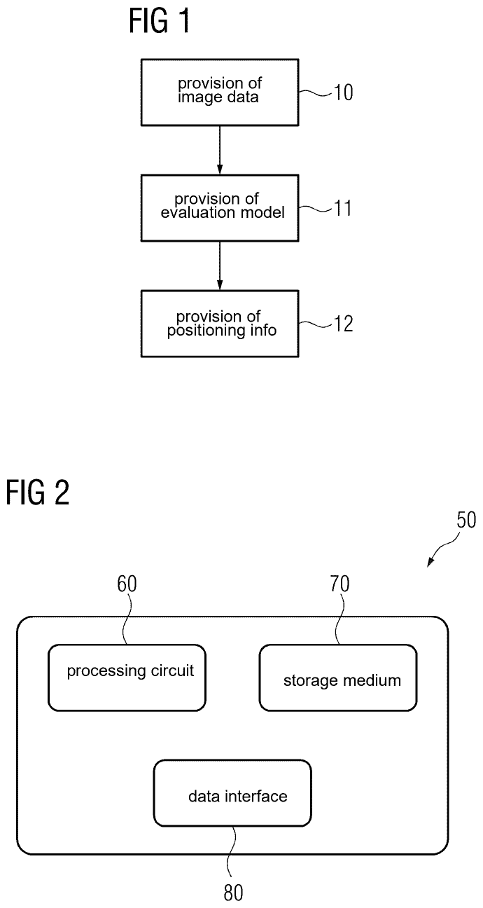

FIG. 1 shows a flow diagram of an exemplary aspect of a computer-assisted method for provision of positioning information for an examination protocol for the examination of a patient. In a first step 10, image data of an overview recording of a patient is provided. In a second step 11, an evaluation model is provided, which is configured, based on the image data provided, to provide positioning information for an examination protocol for the examination of the patient. In a further step 12, positioning information for an examination protocol for the examination of the patient is provided by application of the evaluation model and the image data provided.

FIG. 2 shows an exemplary aspect of a disclosed system 50 for provision of positioning information for an examination protocol for the examination of a patient. The system 50 comprises a processing circuit 60, a storage medium 70, and a data interface 80. The storage medium 70 comprises a computer program with instructions that, when the program is executed, cause the processing circuit 60 to carry out a disclosed method, wherein the data interface 80 is configured to receive the image data of the overview recording of the patient for the evaluation model.

An especially preferred aspect of a disclosed method and system is described below. In the preferred aspect of the disclosed method, positioning information for an examination protocol for the examination of the patient, as explained below, is provided. The preferred aspect of the disclosed method relates in this case to distinguishing a doubled anatomy of a patient, here, for example, two knees of a patient. The disclosed method described below enables it to be established on which of the two knees the examination protocol is to be positioned.

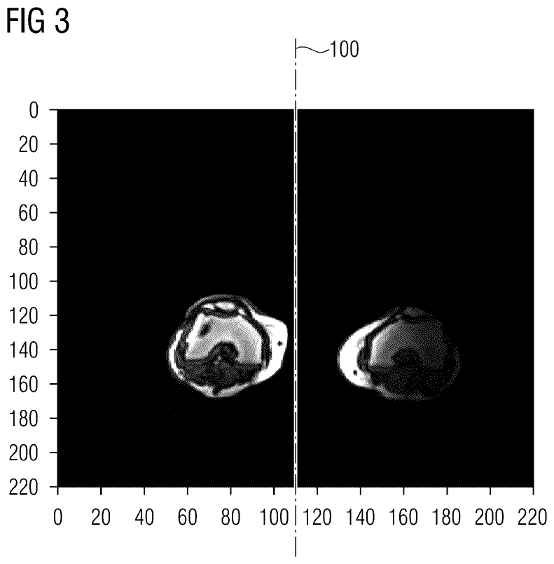

In a first step, an overview recording of a patient, a so-called scout recording, is created and provided as image data. The result is a 3D data volume, which comprises the doubled anatomies, here two knees. Shown in FIG. 3 is an example of an axial section through the image data, in which both knees are comprised. Drawn in FIG. 3 is the center line 100, for example, the Z axis of a computer tomography device. As can be easily seen in FIG. 3, the location/arrangement of the two knees is not symmetrical about the center line 100. Such location of the two knees is deliberately undertaken by the operator, since the knee to be examined is to be located more in the isocenter, i.e. closer to the center line 100.

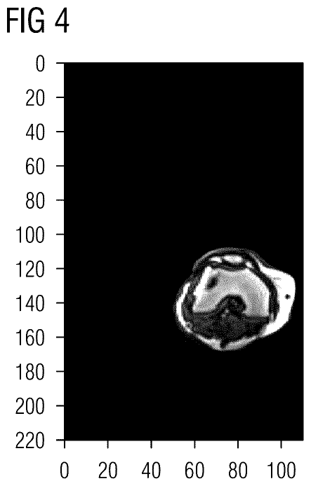

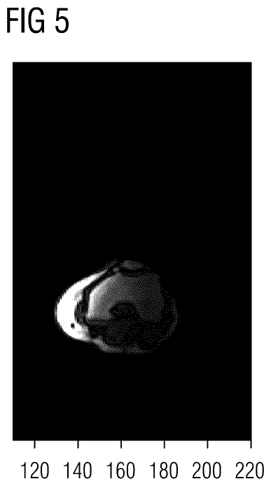

In a further step, the image data of the overview recording is divided with regard to the center line 100 into image data of a first region and image data of a second region. In other words, the image data is divided into two sub-volumes: left and right. In one example, the image data of the overview recording, in this case, is not divided directly at the center line 100 as a reference axis, but with an overlapping area. The overlapping area can in this case amount to between 5 and 30 cm, preferably to between 5 and 20 cm, and especially preferably to between 8 and 12 cm. The overlapping area is preferably chosen so that in the first region, i.e., in the sub-volume on the left, a first anatomy, here the left knee, and in the second region, i.e., in the sub-volume on the right, a second anatomy, here the right knee, is arranged. FIG. 4 in this case shows the sub-volume on the right, i.e., the right knee, and FIG. 5 shows the sub-volume on the left, i.e., the left knee.

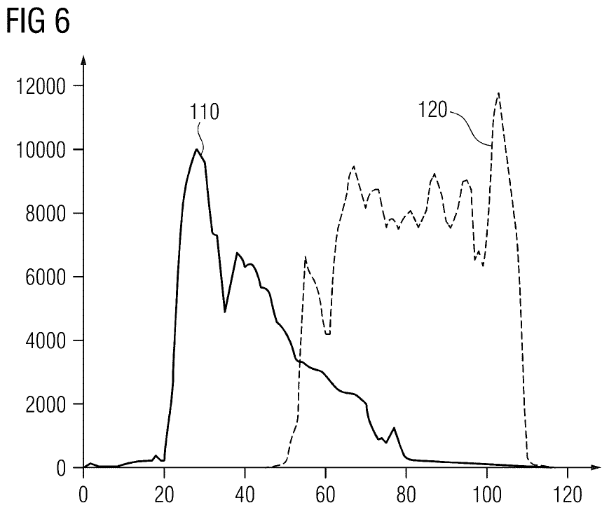

As can be seen in FIGS. 4 and 5, both regions contain a high proportion of so-called empty pixels, i.e., pixels that do not represent any tissue or only contain a noise signal. In order to eliminate the influence of these empty pixels, the image data of both regions is projected onto the X axis, and the projection data shown in FIG. 6 is provided. The projection data in this case represents the respective intensity curve along the X axis. In this case, the projection data of the first region, i.e., of the right knee, is labeled with the reference number 110, and the projection data of the second region, i.e., of the left knee, is labeled with the reference number 120. The example shown in this case relates to one slice, wherein a volume view, of which more later, is also possible.

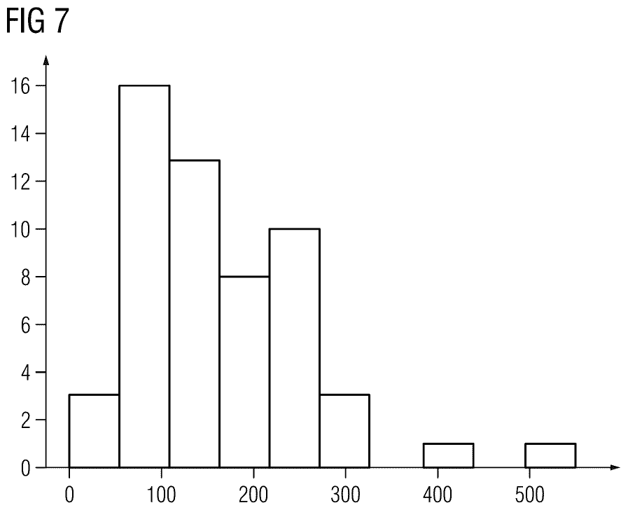

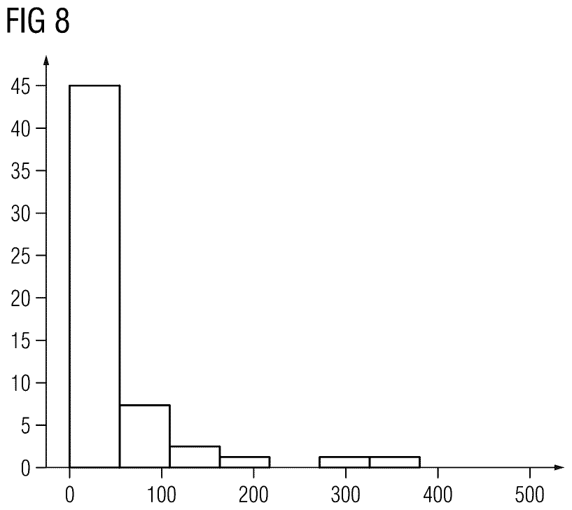

In a further step, corresponding first histogram data and second histogram data are established for the projection data of the first region and the second region, wherein the histogram data is preferably gray value histogram data. The histogram data provides the distribution of the respective brightness values of the two regions or projection data. Where the histogram data is to be visualized, said data can be visualized in the form of bar diagrams, for example. On the horizontal axis in said diagrams, in this case, the gray values are listed, and in parallel to the vertical axis, the frequencies of the respective brightness values are listed. In FIG. 7, in this case, the histogram data for the first region, i.e., for the right knee, is visualized. In FIG. 8, the histogram data for the second region, i.e., for the left knee, is visualized.

Finally, the respective focuses of the histogram data are established in order for these to be employed for the provision of the positioning information for the examination protocol, in that the two focuses of the histogram data are compared with one another. In one example, the first focus of the first histogram data and the second focus of the second histogram data are established by the following formula:

$$S = 1 / n {\sum }_{k = 1}^{n} {W}_{k} * {B}_{k} ;$$

wherein S represents the focus of the histogram data, n represents total number of entries in the histogram data, Bₖ represents number of entries in the bin k, and Wₖ represents value of the bin k.

In the example shown, a first focus for the first region, i.e., for the right knee, of 165 and a second focus for the second region, i.e., for the left knee, of 53 is produced for the histogram data shown in FIGS. 7 and 8. The result of this is that the right knee shown in FIG. 4 is arranged closer to the center line 110 as reference axis, and thus more in the isocenter. Thus, the right knee shown in FIG. 4 can be specified as positioning information for the examination protocol as lateral information, and the examination protocol can be positioned on the right knee.

The example shown in the figures evaluates image data of one slice of the overview recording in this case. As an alternative, the 3D image data can also be evaluated. In this case, only the projection of the data into one plane, along the slice stack direction, is to be provided as an additional step. Such a projection can, however, lead to an image that is unsharp to the human eye, but this projection data can also be processed as described above, so that here too, corresponding focuses of the respective histogram data can be provided.

It is also possible to apply the method to so-called two-stage scouts, for example, for the elbow and hand/wrist. Here, a first Scout recording can initially be provided with a large field of view in order to establish on which side of an anatomy an MRT coil is arranged. Subsequently, on the side established, a high-resolution scout recording with a smaller field of view can be made.

In summary, the present method in particular enables an improved recognition of the laterality to be provided, in that a histogram-based evaluation of the image data of an overview recording of a patient is undertaken. This laterality recognition can be used in order to be able to undertake an automated positioning of an examination protocol.

In addition, it is pointed out that the terms “comprising” and “having” do not exclude other elements or steps, and the indefinite article “a” or “an” does not exclude a plurality. It is further pointed out that features or steps that have been described with reference to above forms of aspect can also be used in combination with other features.

What is more, it is pointed out that, independent of the grammatical term usage of a specific person-related term, individuals with male, female, or other gender identities should be included within the term.

1. A computer-assisted method for generating positioning information for an examination protocol for the examination of a patient, comprising:
providing image data of an overview recording of a patient;
providing an evaluation model configured, based on the image data, to generate positioning information for an examination protocol for the examination of the patient; and
generating positioning information for the examination protocol by applying the evaluation model to the image data,
wherein the positioning information is usable in the examination protocol for the examination of the patient.

2. The computer-assisted method as claimed in claim 1, wherein the evaluation model is further configured to evaluate the image data for an asymmetrical arrangement of the patient in relation to a reference axis or to a reference plane, to generate the positioning information for the examination protocol.

3. The computer-assisted method as claimed in claim 2, wherein the reference axis is a Z axis of a magnetic resonance tomography device or a computed tomography device.

4. The computer-assisted method as claimed in claim 2, wherein the evaluation model is further configured to divide the image data of the overview recording with regard to the reference axis or reference plane into image data of a first region and image data of a second region.

5. The computer-assisted method as claimed in claim 4, wherein the evaluation model is further configured to generate first projection data of the image data of the first region on an X axis and second projection data of the image data of the second region on the X axis.

6. The computer-assisted method as claimed in claim 5, wherein the evaluation model is further configured to generate first histogram data of the first projection data and second histogram data of the second projection data.

7. The computer-assisted method as claimed in claim 6, wherein the evaluation model is further configured to generate first focus data of the first histogram data and second focus data of the second histogram data.

8. The computer-assisted method as claimed in claim 6, wherein the evaluation model is further configured to generate a first focus of the first histogram data and a second focus of the second histogram data through the following formula:
$$S = \frac{1}{n {\sum }_{k = 1}^{n} {W}_{k} * {B}_{k}} ,$$
wherein S represents the focus of the histogram data, n represents total number of entries in the histogram data, Bₖ represents number of entries in bin k, and Wₖ represents value of the bin k.

9. The computer-assisted method as claimed in claim 7, wherein the evaluation model is further configured to generate the positioning information for the examination protocol at least in part on a comparison of the first focus and the second focus.

10. The computer-assisted method as claimed in claim 1, wherein the evaluation model comprises at least one trained algorithm configured, at least based on the image data, to generate the positioning information.

11. The computer-assisted method as claimed in claim 1, wherein the positioning information comprises at least lateral information for an anatomy of a patient that is present twice.

12. The computer-assisted method as claimed in claim 1, wherein the method further comprises:
generating control data for control of a magnetic resonance tomography device or a computed tomography device based on the positioning information.

13. A system for generating positioning information for an examination protocol for examination of a patient, comprising:
a processing circuit;
a non-transitory storage medium; and
a data interface,
wherein the non-transitory storage medium comprises a computer program with instructions that, when the program is executed, cause the processing circuit to carry out the method as claimed in claim 1, wherein the data interface is configured to receive the image data of the overview recording of the patient.

14. A non-transitory computer program element comprising instructions that, when executed on computer devices of a computer environment, are configured to carry out steps of a method for generating positioning information for an examination protocol for the examination of a patient in a system as claimed in claim 13, the method including providing image data of an overview recording of a patient, providing an evaluation model configured, based on the image data, to generate positioning information for an examination protocol for the examination of the patient, and generating positioning information for the examination protocol by applying the evaluation model to the image data, wherein the positioning information is usable in the examination protocol for the examination of the patient.
